(19) United States
(12) Patent Application Publication
(10) Pub. No.: US 2022/0270247 A1
(43) Pub. Date: Aug. 25, 2022

(54) APPARATUS FOR MOVING A MEDICAL OBJECT AND METHOD FOR PROVIDING A CONTROL INSTRUCTION

(71) Applicant: Siemens Healthcare GmbH, Erlangen (DE)

(72) Inventors: Michael Wiets, Langensendelbach (DE); Andreas Meyer, Bubenreuth (DE); Christian Kaethner, Forchheim (DE)

(21) Appl. No.: 17/673,369

(22) Filed: Feb. 16, 2022

(30) Foreign Application Priority Data
Feb. 24, 2021 (DE) 10 2021 201 729.0

(51) Int. Cl. G06T 7/00; G06T 7/70; G06F 3/01; G06T 7/60; G06T 19/00; A61B 34/00; A61B 34/37; A61B 90/00

(52) U.S. Cl. CPC G06T 7/0012; G06T 7/70; G06F 3/017; G06T 7/60; G06T 19/006; A61B 34/25; A61B 34/37; A61B 90/37; G06T 2207/20081; G06T 2200/24; A61B 2034/105

(57) ABSTRACT

An apparatus for moving a medical object includes a movement apparatus and a user interface. The apparatus is configured to receive dataset of the examination region and receive and/or determine positioning information about a positioning of the predefined section. The user interface is configured to display a graphic display of the predefined section with regard to the examination region, and to acquire a user input with regard to the graphic display, which specifies a target positioning and/or movement parameter for the predefined section. The apparatus is configured to determine a control instruction based on the user input, and the movement apparatus is configured to move the medical object in accordance with the control instruction.

The present patent document claims the benefit of German Patent Application No. 10 2021 201 729.0, filed Feb. 24, 2021, which is hereby incorporated by reference in its entirety.

### TECHNICAL FIELD

The disclosure relates to an apparatus for moving a medical object, to a system, to a method for providing a control instruction, to a method for providing a trained function, and to a computer program product.

### BACKGROUND

Frequently interventional medical procedures in or by way of a vascular system of an examination object require introduction, in particular percutaneous introduction, of a, (e.g., elongated), medical object into the vascular system. It may further be necessary, for successful diagnostics and/or treatment, to guide at least a part of the medical object through to a target region to be treated in the vascular system. In such cases the medical object may be moved manually and/or robotically, in particular at a proximal section. Frequently the medical object is moved while using, in particular continuous, X-ray fluoroscopy control. The disadvantage with a manual movement of the medical object is frequently the increased radiation load on the medical operating personnel who are holding the medical object, in particular at the proximal section. With a robotic movement of the medical object, frequently only the operating parameters of a robot holding the proximal section of the medical object may be predetermined by the operating personnel, for example, by a joystick and/or a keyboard. Monitoring and/or adjusting these operating parameters, in particular as a function of a spatial positioning at that moment of the distal end area of the medical object influenced by the robotically guided movement may be the responsibility of the medical operating personnel here.

### SUMMARY AND DESCRIPTION

The underlying object of the disclosure is therefore to make possible an improved control of a predefined section of a robotically moved medical object.

The scope of the present disclosure is defined solely by the appended claims and is not affected to any degree by the statements within this summary. The present embodiments may obviate one or more of the drawbacks or limitations in the related art.

In a first aspect, the disclosure relates to an apparatus for moving a medical object. In this case, the apparatus has a movement apparatus for robotic movement of the medical object and a user interface. Further, in an operating state of the apparatus, at least one predefined section of the medical object is arranged in an examination region of an examination object. The apparatus is embodied to receive a dataset having an image and/or a model of the examination region. The apparatus is further embodied to receive and/or to determine positioning information about a spatial positioning of the predefined section of the medical object. Moreover, the user interface is embodied to display a graphic display of the predefined section of the medical object with regard to the examination region based on the dataset and the positioning information. Furthermore, the user interface is embodied to acquire a user input with regard to the graphic display. In this case the user input specifies a target positioning and/or a movement parameter for the predefined section. Moreover, the apparatus is embodied to determine a control instruction based on the user input. The movement apparatus is further embodied to move the medical object in accordance with the control instruction.

In this case the medical object may be embodied as a, (e.g., elongated), surgical and/or diagnostic instrument. In particular, the medical object may be flexible and/or rigid at least in sections. The medical object may be embodied as a catheter and/or endoscope and/or guide wire.

The examination object may be a human patient and/or an animal patient and/or an examination phantom, in particular a vessel phantom. The examination region may further describe a spatial section of the examination object, which may include an anatomical structure of the examination object, in particular a hollow organ. In this case, the hollow organ may be embodied as a vessel section, in particular an artery and/or vein, and/or as a vessel tree and/or a heart and/or a lung and/or liver.

Advantageously, the movement apparatus may be a robotic apparatus, which is embodied for remote manipulation of the medical object, for example, a catheter robot. Advantageously, the movement apparatus is arranged outside of the examination object. The movement apparatus may further have an, in particular movable and/or drivable, fastening element. Moreover, the movement apparatus may have a cassette element, which is embodied for accommodating at least a part of the medical object. Furthermore, the movement apparatus may have a movement element, which is fastened to the fastening element, for example a stand and/or robot arm. Moreover, the fastening element may be embodied to fasten the movement element to a patient support apparatus. The movement element may further advantageously have at least one actuator element, for example, an electric motor, which is able to be controlled by a provision unit. Advantageously, the cassette element may be able to be coupled, in particular mechanically and/or electromagnetically and/or pneumatically, to the movement element, in particular to the at least one actuator element. In this case, the cassette element may further have at least one transmission element, which is able to be moved by the coupling between the cassette element and the movement element, in particular the at least one actuator element. In particular, the at least one transmission element may be movement-coupled to the at least one actuator element. Advantageously, the transmission element is embodied to transmit a movement of the actuator element to the medical object in such a way that the medical object is moved in a longitudinal direction of the medical object and/or that the medical object is rotated about its longitudinal direction. The at least one transmission element may have a caster and/or roller and/or plate and/or shear plate, which is embodied for transmitting a force to the medical object. The transmission element may further be embodied to hold the medical object, in particular in a stable manner, by transmission of the force. The holding of the medical object may include a positioning of the medical object in a fixed position relative to the movement apparatus.

Advantageously, the movement element may have a number of, in particular independently controllable, actuator elements. The cassette element may further have a number of transmission elements, in particular at least one movement-coupled transmission element for each of the actuator elements. This enables an, in particular independent and/or simultaneous, movement of the medical object along different degrees of freedom of movement to be made possible.

The medical object may, in the operating state of the apparatus, advantageously be introduced by an introduction port at least partly into the examination object in such a way that the predefined section of the medical object is arranged within the examination object, in particular in the examination region and/or hollow organ. The predefined section may describe an, in particular distal, end region of the medical object, in particular a tip. The predefined section may advantageously be predetermined as a function of the medical object and/or of the examination region and/or be defined by a user, in particular by one of the medical operating personnel.

The apparatus may further have a provision unit, which is embodied for controlling the apparatus and/or its components, in particular the movement apparatus. The apparatus, in particular the provision unit, may be embodied for receiving the dataset. Moreover, the apparatus, in particular the provision unit, may be embodied to receive the positioning information. In this case, the receipt of the dataset and/or the positioning information may include an acquisition and/or readout of a computer-readable data memory and/or a receipt from a data storage unit, for example a database. The apparatus, in particular the provision unit, may further be embodied to receive the dataset and/or the positioning information for acquiring the spatial positioning of the predefined section, in particular at that moment from a positioning unit and/or from a medical imaging device. Alternatively, or additionally, the apparatus may be embodied to determine the positioning information, in particular based on the dataset. Advantageously, the apparatus, in particular the provision unit, may be embodied for repeated, in particular continuous, receipt of the dataset and/or of the positioning information.

The positioning information may advantageously include information about a spatial position and/or alignment and/or pose of the predefined section in the examination region of the examination object, in particular at that moment. In particular, the positioning information may describe the spatial positioning of the predefined section, in particular at that moment, with regard to the movement apparatus. The spatial positioning of the predefined section in this case may be described by a length dimension along the longitudinal direction of the medical object and/or by an angle of the medical object relative to the movement apparatus. Alternatively, or additionally, the positioning information advantageously describes the information about the spatial positioning of the predefined section in a patient coordinate system, in particular at that moment.

The dataset may advantageously have an, in particular time-resolved, two-dimensional (2D) and/or three-dimensional (3D) image of the examination region, in particular of the hollow organ. In particular, the dataset may have a contrasted and/or segmented image of the examination region, in particular of the hollow organ. The dataset may further map the examination region preoperatively and/or intraoperatively. Alternatively, or additionally, the dataset may have a 2D and/or 3D model, in particular a central line model and/or a volume model, (e.g., a volume mesh model), of the examination region, in particular of the hollow organ. The dataset may advantageously be registered with the patient coordinate system and/or with regard to the movement apparatus.

The user interface may advantageously have a display unit and an acquisition unit. In this case, the display unit may be at least partly integrated into the acquisition unit or vice versa. Advantageously, the apparatus may be embodied to create the graphic display of the predefined section based on the dataset and the positioning information. The user interface, in particular the display unit, may further be embodied to display the graphic display of the predefined section of the medical object with regard to the examination region based on the dataset and the positioning information. In this case, the graphic display of the predefined section may advantageously have an, in particular real and/or synthetic, image and/or an, in particular abstracted, model of the predefined section of the medical object. Moreover, the graphic display may have an, in particular real and/or synthetic, image and/or a model of at least one section of the examination region, in particular of the hollow organ. The display unit may advantageously be embodied to display the graphic display spatially resolved two-dimensionally and/or three-dimensionally. The display unit may further be embodied to display the graphic display in a time-resolved manner, for example as a video and/or scene. Moreover, the apparatus may be embodied to adjust the graphic display, in particular in real time, for a change in the positioning information and/or the dataset. In particular, the apparatus may be embodied to create the graphic display having an, in particular weighted, overlaying of the image and/or of the model of the examination region with an, in particular synthetic, image and/or a model of the predefined section based on the positioning information.

Furthermore, the user interface, in particular the acquisition unit, may be embodied to acquire the user input with regard to the graphic display. In this case, the acquisition unit may have an input device, (e.g., a computer mouse and/or a touchpad and/or a keyboard), and/or be embodied for acquiring an, in particular external, input, (e.g., a pointing device, in particular a stylus), and/or part of a user's body, (e.g., a finger). For this, the acquisition unit may include an optical and/or haptic and/or electromagnetic and/or acoustic sensor, for example a camera, in particular a mono and/or stereo camera, and/or a touch-sensitive surface. In this case, the acquisition unit may be embodied to acquire a spatial positioning of the external input, in particular in a time-resolved manner, in particular with regard to the graphic display.

Advantageously, the user interface may be embodied to associate the user input spatially and/or temporally with the graphic display of the predefined section, in particular a pixel and/or image area of the graphic display. In this case, the user input may specify a target positioning and/or a movement parameter for the predefined section of the medical object. The target positioning may predetermine a spatial position and/or alignment and/or pose, which the predefined section of the medical object is to assume. The movement parameter may predetermine a direction of movement and/or a speed for the predefined section. Furthermore, the apparatus may be embodied to associate the user input with anatomical and/or geometrical features of the dataset. The anatomical features may include an image and/or a model of the hollow organ and/or an adjoining tissue and/or an anatomical landmark, for example an ostium and/or a bifurcation. The geometrical features may further include a contour and/or a contrast gradation.

Advantageously, the apparatus, in particular the provision unit, may be embodied to determine the control instruction based on the user input. In this case, the control instruction may include at least one command for an, in particular step-by-step, control of the movement apparatus. In particular, the control instruction may include at least one command, in particular a temporal series of commands for specifying an, in particular simultaneous, translation and/or rotation of the medical object, in particular of the predefined section, by the movement apparatus. Advantageously, the provision unit may be embodied to translate the control instruction and to control the movement apparatus based thereon. Moreover, the movement apparatus may be embodied to move the medical object based on the control instruction, in particular translationally and/or rotationally. Furthermore, the movement apparatus may be embodied to deform the predefined section of the medical object in defined way, for example, by a cable within the medical object. The apparatus may be embodied additionally to determine the control instruction based on the positioning information for spatial positioning of the predefined section of the medical object, in particular at that moment.

The proposed apparatus may make possible an improved, in particular intuitive, control of a movement of the predefined section of the medical object by a user. In particular, the proposed apparatus may make possible an, in particular direct, control of the movement of the predefined section with regard to the graphic display of the predefined section with regard to the examination region.

In a further embodiment, the dataset may further have an image and/or a model of the predefined section. In this case, the apparatus may further be embodied to determine the positioning information based on the dataset.

The dataset may advantageously include medical image data recorded by a medical imaging device. In this case the medical imaging data may have an, in particular intraoperative, image of the predefined section in the examination area. The image of the predefined section may further be spatially resolved two-dimensionally and/or three-dimensionally. Moreover, the image of the predefined section may be time-resolved. Advantageously the apparatus may be embodied to receive the dataset, in particular the medical image data, in particular in real time, from the medical imaging device. Advantageously the dataset, in particular the medical image data, may be registered with the patient coordinate system and/or the movement apparatus.

Alternatively, or additionally, the dataset may have an, in particular 2D and/or 3D, model of the predefined section. The model may advantageously represent the predefined section realistically, (e.g., as a volume mesh model), and/or in an abstracted way, (e.g., as a geometrical object).

Advantageously, the apparatus may be embodied to localize the predefined section in the dataset, in particular in the medical image data. In this case, the localization of the predefined section in the dataset may include an identification, for example, a segmentation of pixels of the dataset, in particular of the medical image data, with the pixels mapping the predefined section. In particular, the apparatus may be embodied to identify the predefined section in the dataset based on a contour and/or marker structure of the predefined section. Moreover, the apparatus may be embodied to localize the predefined section with regard to the patient coordinate system and/or in relation to the movement apparatus based on the dataset, in particular because of its registration. Moreover, the apparatus may be embodied, in particular in addition to the spatial position of the predefined section, to determine an alignment and/or pose of the predefined section based on the dataset. For this, the apparatus may be embodied to determine a spatial course of the predefined section based on the dataset.

Advantageously, the positioning information for, in particular instantaneous, spatial positioning of the predefined section of the medical object may inherently be registered with the dataset and/or the graphic display.

In a further embodiment, the apparatus may be embodied to determine the control instruction having an instruction for a forward movement and/or backward movement and/or rotational movement of the medical object based on the user input.

The forward movement may describe a movement of the medical object directed away from the movement apparatus, in particular distally. The backward movement may further describe a movement of the medical object directed towards the movement apparatus, in particular proximally. The rotational movement may describe a rotation of the medical object about its longitudinal direction.

The apparatus may be embodied to determine the control instruction having an instruction for a series of part movements and/or a movement of the medical object composed of a number of part movements based on the user input. In this case the part movements may in each case include a forward movement and/or backward movement and/or rotational movement of the medical object. Moreover, the movement parameters of the respective part movements may be different, for example, a speed of movement and/or a direction of movement and/or a movement duration and/or a movement distance and/or an angle of rotation.

The proposed form of embodiment may advantageously make it possible to translate the user input, which specifies the target positioning and/or the movement parameters for the predefined section, into a control instruction for the movement apparatus, which is arranged in particular at a proximal section of the medical object.

In a further embodiment, the user interface may be embodied to acquire the user input repeatedly and/or continuously. In this case, the apparatus may further be embodied to determine and/or adjust the control instruction based on the last user input acquired in each case.

Advantageously, the user interface may be embodied to associate the last user input acquired in each case spatially and/or temporally with the graphic display of the predefined section, in particular the last one displayed, in particular a pixel and/or image region of the graphic display.

This makes it possible for a movement of the medical object, in particular of the predefined section, advantageously to be controlled by the user input in real time.

In a further embodiment, the user interface may be embodied to acquire the user input including an input at a single point and/or an input gesture.

In this case, the input at a single point may be regarded as a spatially and/or temporally isolated input event at the user interface. The input gesture may further be regarded as a spatially and temporally resolved input event at the user interface, for example, a swipe movement.

Advantageously, the apparatus may be embodied to determine the control instruction as a function of a form of the user input. In particular, the apparatus may be embodied to identify a user input including an input at a single point as a specification of a target positioning for the predefined section. Moreover, the apparatus may be embodied to identify a user input including an input gesture as a specification of a movement parameter for the predefined section.

The user interface may further be embodied to acquire a further user input, in particular including a further input at a single point and/or a further input gesture. In this case, the apparatus, in particular the provision unit, may be embodied to adjust the graphic display as a function of the further user input. In particular, the apparatus may be embodied to adjust the graphic display by a scaling, in particular zooming-in and/or zooming-out, and/or windowing and/or a transformation, in particular a rotation and/or translation and/or deformation, of the dataset, in particular with regard to an imaging level and/or direction of view of the graphic display. The adjustment of the graphic display may further include an at least temporary display, for example, an overlaying and/or a display of visual help elements, for example of a warning message and/or of a highlighting of geometrical and/or anatomical features of the dataset.

The proposed form of embodiment may make possible an especially intuitive control of the movement of the medical object, in particular of the predefined section.

In a further embodiment, the user interface may have an input display. In this case, the input display may be embodied to acquire the user input on a touch-sensitive surface of the input display.

Advantageously, the input display may be embodied for, in particular simultaneous, display of the graphic display of the predefined section of the medical object and acquisition of the user input. The input display may advantageously be embodied as a capacitive and/or resistive input display. In this case, the input display may have a touch-sensitive surface, in particular, running flat. Advantageously, the input display may be embodied to display the graphic display of the predefined section on the touch-sensitive surface. Moreover, the provision unit, in particular the touch-sensitive surface, may be embodied for spatially and/or temporally resolved acquisition of the user input, in particular by the input device. This enables the user input advantageously to be inherently registered with the graphic display of the predefined section.

In a further embodiment, the user interface may have a display unit and an acquisition unit. In this case the apparatus may be embodied to create the graphic display as an augmented and/or virtual reality. The display unit may further be embodied to display the augmented and/or virtual reality. Moreover, the acquisition unit may be embodied to acquire the user input with regard to the augmented and/or virtual reality.

The display unit may advantageously be embodied as portable, in particular able to be carried by a user. The display unit may further be embodied for, in particular stereoscopic, display of the augmented and/or virtual reality (abbreviated to AR or VR respectively). In this case, the display unit may be embodied at least partly transparent and/or translucent. Advantageously, the display unit may be embodied in such a way that it is able to be carried by the user at least partly within the field of view of the user. For this, the display unit may advantageously be embodied as a head-mounted unit, in particular head mounted display (HMD), and/or helmet, in particular data helmet, and/or screen.

The display unit may further be embodied to display real objects, (e.g., physical), in particular medical, objects and/or the examination objects, overlaid with virtual data, in particular measured and/or simulated and/or processed medical image data and/or virtual objects and show them in a display, in particular stereoscopically.

Advantageously, the user interface may further have an acquisition unit, which is embodied to acquire the user input. In this case, the acquisition unit may be integrated at least partly into the display unit. This enables an inherent registration between the user input and the augmented and/or virtual reality to be made possible. Alternatively, the acquisition unit may be arranged separately, in particular spatially apart from the display unit. In this case, the acquisition unit may advantageously continue to be embodied for acquisition of a spatial positioning of the display unit. This advantageously enables a registration between the user input and the augmented and/or virtual reality displayed by the display unit to be made possible. Advantageously, the acquisition unit may include an optical and/or haptic and/or electromagnetic and/or acoustic sensor, which is embodied for acquiring the user input, in particular within the field of view of the user, (e.g., a camera, in particular a mono and/or stereo camera). In particular, the acquisition unit may be embodied for two-dimensional and/or three-dimensional acquisition of the user input, in particular based on the input device. The user interface may be further be embodied to associate the user input spatially and/or temporally with the graphic display, in particular the augmented and/or virtual reality.

This enables an especially realistic and/or immersive control of the movement of the medical object, in particular of the predefined section, to be made possible.

In a further embodiment, the dataset may include planning information about movement of the medical object. In this case, the planning information may have at least one first defined area in the dataset. Moreover, the apparatus may be embodied to identify based on the positioning information and of the dataset, whether the predefined section is arranged in the at least one first defined area. In this case, the apparatus may further be embodied, in the affirmative case, to adjust the graphic display and/or to provide a recording parameter at a medical imaging device for recording a further dataset.

The planning information may advantageously include a path planning and/or annotations, in particular with regard to a preoperative image of the examination region in the dataset. Advantageously, the planning information may be registered with the dataset and/or the positioning information and/or the patient coordinate system and/or the movement apparatus. Moreover, the planning information may have at least one first defined area in the dataset. In this case, the at least one first defined area may describe a spatial section of the examination object, in particular a spatial volume and/or a central line section, which may include an anatomical structure of the examination object, in particular a hollow organ and/or an anatomical landmark, (e.g., an ostium and/or a bifurcation), and/or anatomical peculiarity, (e.g., an occlusion, in particular a thrombus and/or a chronic total occlusion (CTO), and/or a stenosis and/or a hemorrhage). Advantageously, the at least one first defined area may have been defined preoperatively and/or intraoperatively by a user input, in particular by the user interface. In particular, the at least one first defined area may include a number of pixels, in particular a spatially coherent set of pixels, of the dataset. Moreover, the planning information may have a number of first defined areas in the dataset.

The apparatus may further be embodied, based on the positioning information and the dataset, in particular through a comparison of spatial coordinates, to identify whether the predefined section is arranged, in particular at that moment, in the at least one first defined area. In particular, the apparatus may be embodied to identify, based on the positioning information and the dataset, whether the predefined section is arranged at least partly within the spatial section of the examination region described by the at least one first defined area in the dataset. Provided the planning information has a number of first defined areas in the dataset, the apparatus may advantageously be embodied to identify whether the predefined section is arranged in at least one of the number of first defined areas in the dataset.

Moreover, the apparatus may be configured, when the predefined section is arranged in the at least one first defined area, to adjust the graphic display, in particular semi-automatically and/or automatically and/or to provide a recording parameter to a medical imaging device for recording a further dataset. In particular, the apparatus may be embodied to adjust the graphic display through a scaling, in particular zooming-in and/or zooming-out, and/or windowing in such a way that the at least one first defined area in which the predefined section is shown at least partly arranged in the operating state of apparatus, in particular completely and/or filling the screen. Furthermore, the adjustment of the graphic display may include a transformation, in particular a rotation and/or translation and/or deformation, of the dataset, in particular in relation to an imaging plane and/or direction of view of the graphic display. Furthermore, the apparatus may be embodied to adjust the graphic display for an approximation of the predefined section to the at least one first defined area and/or for the arrangement of the predefined section at least partly within the at least one first defined area in steps and/or steplessly. Additionally, or alternatively, the apparatus may be embodied, with an at least partial arrangement of the predefined section of the medical object within the at least one first defined area, to output an acoustic and/or haptic and/or optical signal to the user. In particular, the apparatus may be embodied to adjust the graphic display based on a further user input.

Furthermore, the apparatus may be embodied to provide a recording parameter in such a way that an improved image of the predefined section and/or of the at least one first defined area in the further dataset is made possible. The recording parameter may advantageously include an, in particular spatial and/or temporal, resolution and/or recording rate and/or pulse rate and/or dose and/or collimation and/or a recording region and/or a spatial positioning of the medical imaging device, in particular with regard to the examination object and/or with regard to the predefined section and/or in relation to the at least one first defined area. Advantageously, the apparatus may be embodied to determine the recording parameter based on an organ program and/or based on a lookup table, in particular as a function of the at least one first defined area in which the predefined section is arranged at least partly in the operating state of the apparatus. In this case, the medical imaging device for recording the further dataset may be the same as or different from the medical imaging device for recording the dataset. The apparatus may further be embodied to receive the further dataset and to replace the dataset with the further dataset.

The proposed form of embodiment may advantageously make possible an optimization of the graphic display, in particular for a spatial arrangement of the predefined section within the at least one first defined area. This enables an improved, in particular more precise, control of the movement of the predefined section to be made possible.

In a further embodiment, the apparatus may further be embodied to identify geometrical and/or anatomical features in the dataset. Moreover, the apparatus may be embodied, based on the identified geometrical and/or anatomical features, to define at least one second area in the dataset. Moreover, the apparatus may be embodied, based on the positioning information and the dataset, to identify whether the predefined section is arranged in the at least one second defined area. Moreover, the apparatus may be embodied, in the affirmative case, to adjust the graphic display and/or to provide a recording parameter to a medical imaging device for recording a further dataset.

The geometrical features may include lines, in particular contours and/or edges, and/or corners and/or contrast transitions and/or a spatial arrangement of these features. The anatomical features may include anatomical landmarks and/or tissue boundaries, (e.g., a vessel and/or organ wall), and/or anatomical peculiarities, (e.g., a bifurcation and/or a chronic coronary occlusion), and/or vessel parameters, (e.g., a diameter and/or constrictions). In this case, the apparatus may be embodied to identify the geometrical and/or anatomical features based on image values of pixels of the dataset. The apparatus may further be embodied to identify the geometrical and/or anatomical features based on a classification of static and/or moving regions of the examination region in the dataset, for example based on time intensity curves. Moreover, the apparatus may be embodied to identify the geometrical and/or anatomical features in the dataset by a comparison with an anatomy atlas and/or by application of a trained function.

The apparatus may further be embodied to define at least one second area, in particular a number of second areas, in the dataset based on the identified geometrical and/or anatomical features. In this case, the at least one second defined area may describe a spatial section of the examination object, in particular a spatial volume and/or a central line section, which includes at least one of the identified geometrical and/or anatomical features. In particular, the at least one second defined area may include a number of pixels, in particular a spatially coherent set of pixels, of the dataset.

The apparatus may further be embodied to identify based on the positioning information and of the dataset whether the predefined section is arranged in the at least one second defined area, in particular at that moment. In particular, the apparatus may be embodied to identify based on the positioning information and of the dataset whether the predefined section is arranged at least partly within the spatial section of the examination region described by the at least one second defined area in the dataset. Moreover, the apparatus may be embodied to identify whether the predefined section is arranged in at least one of a number of second defined areas in the dataset.

Moreover, the apparatus may be configured, when the arrangement of the predefined section is in the at least one second defined area, to adjust the graphic display, (e.g., semi-automatically and/or automatically), and/or to provide a recording parameter to a medical imaging device for recording a further dataset. In particular, the apparatus may be embodied to adjust the graphic display by a scaling, in particular zooming-in and/or zooming-out, and/or windowing, in such a way that the at least one second defined area, in which the predefined section is at least partly arranged in the operating state of the apparatus, in particular completely and/or filling the screen, is displayed. Moreover, the adjustment of the graphic display may include a transformation, in particular a rotation and/or translation and/or deformation, of the dataset, in particular in relation to an imaging plane and/or direction of view of the graphic display. Moreover, the apparatus may be embodied to adjust the graphic display for an approximation of the predefined section to the at least one second defined area and/or for the arrangement of the predefined section at least partly within the at least one second defined area step-by-step and/or steplessly. Additionally, or alternatively, the apparatus may be embodied, for an at least part arrangement of the predefined section of the medical object within the at least one second defined area, to output an acoustic and/or haptic and/or optical signal to the user. In particular, the apparatus may be embodied to adjust the graphic display based on the further user input.

Furthermore, the apparatus may be embodied to provide a recording parameter in such a way that an improved image of the predefined section and/or of the at least one second defined area in the further dataset is made possible. The recording parameter may advantageously include an, in particular spatial and/or temporal, resolution and/or recording rate and/or pulse rate and/or dose and/or collimation and/or a recording area and/or a spatial positioning of the medical imaging device, in particular in relation to the examination object and/or in relation to the predefined section. Advantageously, the apparatus may be embodied to determine the recording parameter based on an organ program and/or based on a lookup table, in particular as a function of the at least one second defined area in which the predefined section is at least partly arranged in the operating state of the apparatus. In this case, the medical imaging device for recording of the further dataset may be the same as or different from the medical imaging device for recording the dataset. The apparatus may further be embodied to receive the further dataset and to replace the dataset with the further dataset.

The proposed form of embodiment may advantageously make possible an optimization of the graphic display, in particular for a spatial arrangement of the predefined section within the at least one second defined area. This enables an improved, in particular more precise, control of the movement of the predefined section to be made possible.

In a further embodiment, the dataset may include planning information for movement of the medical object. Moreover, the apparatus may be embodied to define the at least one second defined area additionally based on the planning information.

The planning information may have all features and characteristics that are described in relation to another form of embodiment of the proposed apparatus and vice versa. Advantageously, the planning information may have path planning for a positioning and/or movement of the medical object, in particular of the predefined section, along a planned path in the examination area. In this case the apparatus may further be embodied to identify the geometrical and/or anatomical features at least along and/or in a spatial environment of the planned path. Moreover, the apparatus may be embodied to define the at least one second area based on the planning information at least along the planned path in the dataset.

The proposed form of embodiment may advantageously make possible an optimization of the graphic display, taking into account the planning information, in particular along a planned path for the movement of the predefined section.

In a second aspect, the disclosure relates to a system having a medical imaging device and a proposed apparatus for moving a medical object. In this case the medical imaging device is embodied to record a dataset having an image of an examination region of an examination object and provide it to the apparatus.

The advantages of the proposed system may correspond to the advantages of the proposed apparatus. Features, advantages, or alternate forms of embodiment may likewise be transferred to the other claimed subject matter and vice versa.

The medical imaging device may advantageously be embodied as an X-ray device, in particular C-arm X-ray device, and/or magnetic resonance tomograph (MRT) and/or computed tomography system (CT) and/or ultrasound device and/or positron emission tomography system (PET). The system may further have an interface, which is embodied to provide the dataset to the apparatus, in particular to the provision unit. The interface may further be embodied to receive the recording parameters for recording the further dataset. Moreover, the medical imaging device may be embodied to record the further dataset by the received recording parameter and provide it to the apparatus, in particular to the provision unit.

The solution is described below both in relation to methods and apparatuses for providing a control instruction and also in relation to methods and apparatuses for providing a trained function. Features, advantages, and alternate forms of embodiment of data structures and/or functions for methods and apparatuses for providing a control instruction may be transferred here to similar data structures and/or functions for methods and apparatuses for providing a trained function. Similar data structures may be identified here by the prefix “training”. Furthermore, the trained functions used in the methods and apparatuses for providing a control instruction may be adjusted and/or provided by methods and apparatuses for providing a trained function.

In a third aspect, the disclosure relates to a method for providing a control instruction. In a first act, a dataset having an image and/or a model of an examination region of an examination object is received. In this case, the at least one predefined section of a medical object is arranged in the examination area. In a second act, positioning information for a spatial positioning of the predefined section is received and/or determined. In a third act, a graphic display of the predefined section of the medical object in relation to the examination region based on the dataset and the positioning information is shown. In a fourth act, a user input in relation to the graphic display is acquired. In this case, the user input specifies a target positioning and/or a movement parameter for the predefined section. In a fifth act, a control instruction is determined based on the user input. In this case the control instruction has an instruction for control of a movement apparatus. Moreover, the movement apparatus is embodied to hold and/or to move the medical object arranged at least partly in the movement apparatus by transmission of a force in accordance with the control instruction. In a sixth act, the control instruction is provided.

The advantages of the proposed method for providing a control instruction may correspond to the advantages of the proposed apparatus for moving a medical object and/or of the proposed system. Features, advantages, or alternate forms of embodiment mentioned here may likewise be transferred to the other claimed subject matter and vice versa.

The receipt of the dataset and/or the positioning information may include an acquisition and/or readout of a computer-readable data memory and/or a receipt from a data memory unit, for example a database. The dataset and/or the positioning information may further be received from a positioning unit for acquiring the spatial positioning of the predefined section and/or of a medical imaging device, in particular at that moment.

The provision of the control instruction may include storage on a computer-readable memory medium and/or display on a display unit and/or transmission to a provision unit. The provided control instruction may advantageously support a user in the control of the movement apparatus.

In a further embodiment, the dataset may have an image and/or a model of the predefined section. In this case, the positioning information may be determined based on the dataset.

In a further embodiment, the dataset may include planning information for a planned movement of the medical object. In this case the planning information may have at least one first defined area in the dataset. Moreover, based on the positioning information and of the dataset, it may be identified whether the predefined section is arranged in the at least one first defined area. In the affirmative case, the graphic display may be adjusted and/or a recording parameter is provided to a medical imaging device for recording a further dataset.

Advantageously, the further dataset may be recorded by the medical imaging device based on the recording parameter provided. Hereafter, the further dataset may be received and provided for repeated execution of the proposed method as the dataset.

In a further embodiment, the geometrical and/or anatomical features in the dataset may be identified. In this case, based on the identified geometrical and/or anatomical features, at least one second area in the dataset may be defined. Moreover, it may be identified based on the positioning information and the dataset whether the predefined section is arranged in the at least one second defined area. In the affirmative case, the graphic display may be adjusted and/or a recording parameter is provided to a medical imaging device for recording a further dataset.

Advantageously, the further dataset may be recorded by the medical imaging device based on the recording parameters provided. Hereafter, the further dataset may be received and provided for repeated execution of the proposed method as the dataset.

In a further embodiment, the dataset may include planning information for a planned movement of the medical object. In this case, the at least one second area may additionally be defined based on the planning information.

In a further embodiment, the geometrical and/or anatomical features in the dataset may be identified by applying a trained function to input data. In this case, the input data may be based on the dataset. Moreover, at least one parameter of the trained function may be based on a comparison of training features with comparison features.

The trained function may advantageously be trained by a machine learning method. In particular the trained function may be a neural network, in particular a convolutional neural network (CNN) or a network including a convolutional layer.

The trained function maps input data to output data. Here, the output data may continue to depend on one or more parameters of the trained function. The one or more parameters of the trained function may be determined and/or adjusted by training. The determination and/or the adjustment of the one or more parameters of the trained function may be based on a pair including training input data and associated training output data, in particular comparison output data, wherein the trained function is applied to the training input data to create training mapping data. In particular, the determination and/or the adjustment may be based on a comparison of the training mapping data and the training output data, in particular the comparison output data. A trainable function, meaning a function with one or more parameters not yet adjusted, may be referred to as a trained function.

Other terms for trained function are trained mapping specification, mapping specification with trained parameters, function with trained parameters, algorithm based on artificial intelligence, machine learning algorithm. An example of a trained function is an artificial neural network, wherein the edge weights of the artificial neural network correspond to the parameters of the trained function. Instead of the term “neural network,” the term “neural net” may also be used. In particular, a trained function may also be a deep neural network or deep artificial neural network. A further example of a trained function is a Support Vector Machine. Furthermore, other machine learning algorithms are able to be employed, in particular, as the trained function.

The trained function may be trained in particular by back propagation. First of all, training mapping data may be determined by application of the trained function to training input data. Hereafter, a deviation between the training mapping data and the training output data, in particular the comparison output data, may be established by using an error function on the training mapping data and the training output data, in particular the comparison output data. At least one parameter, in particular a weighting, of the trained function, in particular of the neural network, based on a gradient of the error function in relation to the at least one parameter of the trained function may further be iteratively adjusted. This enables the deviation between the training mapping data and the training output data, in particular the comparison output data, advantageously to be minimized during the training of the trained function.

Advantageously, the trained function, in particular the neural network, has an input layer and an output layer. In this case, the input layer may be embodied for receiving input data. The output layer may further be embodied for providing mapping data. In this case, the input layer and/or the output layer may each include a number of channels, in particular neurons. Advantageously, the trained function may have an encoder-decoder architecture.

At least one parameter of the trained function may be based on a comparison of the training features with the comparison features. In this case, the training features and/or the comparison features may advantageously be provided as a part of a proposed computer-implemented method for providing a trained function, which will be explained in the further course of the description. In particular, the trained function may be provided by a form of embodiment of the proposed computer-implemented method for providing a trained function.

In a further embodiment, the input data may additionally be based on the positioning information.

Advantageously, this enables a higher computing efficiency in the identification of the geometrical and/or anatomical features in the dataset to be achieved by the application of the trained function to the input data. Advantageously the trained function may be embodied to identify the geometrical and/or anatomical features in the dataset locally and/or regionally, in particular not globally, based on the positioning information.

In a fourth aspect, the disclosure relates to a, (e.g., computer-implemented), method for providing a trained function. In a first act, a training dataset having an image and/or a model of a training examination area of a training examination object is received. In a second act, comparison features in the training dataset are identified. In a third act, training features are identified by application of the trained function to input data. In this case the input data is based on the training dataset. In a fourth act, at least one parameter of the trained function is adjusted by a comparison of the training features with the comparison features. In a fifth act, the trained function is provided.

The receipt of the training dataset may include an acquisition and/or readout of a computer-readable data memory and/or a receipt from a data memory unit, for example, a database. The training dataset may further be provided by a provision unit of a medical imaging device. In this case, the medical imaging device may be the same as or different from the medical imaging device for recording the dataset. Moreover, the training dataset may be simulated. The training dataset may further in particular have all characteristics of the dataset, which have been described in relation to the apparatus for moving a medical object and/or the method for providing a control instruction and vice versa.

The training examination object may be a human and/or animal patient. The training examination object may further advantageously be different from or the same as the examination object that has been described in relation to the apparatus for moving a medical object and/or to the method for providing a control instruction. In particular, the training dataset may be received for a plurality of different training examination objects. The training examination area may have all characteristics of the examination region, which have been described in relation to the apparatus for moving a medical object and/or to the method for providing a control instruction and vice versa.

The identification of comparison features in the training dataset may include an, in particular manual and/or semi-automatic and/or automatic, annotation. Moreover, the comparison features may be identified by application of an algorithm for pattern recognition and/or by an anatomy atlas. The comparison features may advantageously include geometrical and/or anatomical features of the training examination object, which are mapped in the training dataset. Moreover, the identification of the comparison features in the training dataset may include an identification of at least one marker structure in the examination area, for example a stent marker.

The training features may advantageously be created by application of the trained function to the input data. In this case the input data may be based on the training dataset. The comparison between the training features and the comparison features further enables the at least one parameter of the trained function to be adjusted. In this case, the at least one parameter of the trained function may advantageously be adjusted in such a way that a deviation between the training features and the comparison features is minimized. The adjustment of the at least one parameter of the trained function may include an optimization, in particular minimization, of a cost value of a cost function, wherein the cost function characterizes the deviation between the training features and the comparison features. In particular the adjustment of the at least one parameter of the trained function may include a regression of the cost value of the cost function.

The provision of the trained function may include a storage on a computer-readable memory medium and/or a transmission to a provision unit. Advantageously, the trained function provided may be used in a form of embodiment of the proposed method for providing a control instruction.

In a further embodiment, positioning information for a spatial positioning of a predefined section of a medical object may be received. In this case, the predefined section may be arranged in the training examination area. Moreover, the input data may additionally be based on the training positioning information.

The training positioning information may have all characteristics of the positioning information, which have been described in relation to the apparatus for moving a medical object and/or the method for providing a control instruction and vice versa.

The receipt of the training positioning information may include an acquisition and/or readout of a computer-readable data memory and/or a receipt from a data memory unit, for example a database. Moreover, the training positioning information may be received from a positioning unit for acquiring the, in particular current, spatial positioning of the predefined section and/or from the medical imaging device. As an alternative, the training positioning information may be simulated.

Advantageously, the comparison features in the training dataset may additionally be identified based on the training positioning information. In particular, the comparison features in the training dataset may be identified locally and/or regionally, for example, within a predefined distance around the spatial positioning of the predefined section described by the training positioning information and/or along a longitudinal direction of the medical object.

Advantageously, the input data of the trained function may additionally be based on the training positioning information. Moreover, the trained function may advantageously be embodied to identify the geometrical and/or anatomical training features in the training dataset locally and/or regionally, in particular not globally, based on the training positioning information.

The disclosure may further relate to a training unit, which has a training computing unit, a training memory unit, and a training interface. In this case, the training unit may be embodied for carrying out a form of embodiment of the proposed method for providing a trained function, by the components of the training unit being embodied to carry out the individual method acts.

The advantages of the proposed training unit may correspond to the advantages of the proposed method for providing a trained function. Features, advantages, or alternate forms of the embodiments mentioned here may likewise also be transferred to the other claimed subject matter and vice versa.

In a fifth aspect, the disclosure relates to a computer program product with a computer program, which is able to be loaded directly into a memory of a provision unit, with program sections for carrying out all acts of the computer-implemented method for providing a control instruction and/or one of its aspects when the program sections are executed by the provision unit; and/or which is able to be loaded directly into a training memory of a training unit, with program sections for carrying out all acts of the computer-implemented method for providing a trained function and/or one of its aspects when the program sections are executed by the training unit.

The disclosure may further relate to a computer-readable memory medium, on which program sections able to be read and executed by a provision unit are stored for executing all acts of the method for providing a control instruction and/or one of its aspects when the program sections are executed by the provision unit; and/or on which program sections able to be read and executed by a training unit are stored for executing all acts of the method for providing a trained function and/or one of its aspects when the program sections are executed by the training unit.

The disclosure may further relate to a computer program or computer-readable storage medium including a trained function provided by a proposed computer-implemented method or one of its aspects.

A software-based realization may have the advantage that the provision units and/or training units already used may be upgraded in a simple way by a software update in order to work in the ways disclosed herein. Such a computer program product, along with the computer program, may include additional elements, such as documentation and/or additional components, as well as hardware components, such as hardware keys (e.g., dongles, etc.) for using the software.

### BRIEF DESCRIPTION OF THE DRAWINGS

Exemplary embodiments are shown in the drawings and are described in more detail below. In different figures the same reference characters are used for the same features. In the figures:

FIG. 1 depicts a schematic diagram of an example of an apparatus for moving a medical object.

FIG. 2 depicts a schematic diagram of an example of a system.

FIG. 3 depicts a schematic diagram of an example of a movement apparatus.

FIG. 4 depicts a schematic diagram of an example of a user interface in a form of embodiment as a touch-sensitive input display.

FIG. 5 depicts a schematic diagram of an example of a user interface embodied to display an augmented and/or virtual reality.

FIG. 6 to 11 depict schematic diagrams of different forms of embodiments of a method for providing a control instruction.

FIGS. 12 and 13 depict schematic diagrams of different forms of embodiments of a method for providing a trained function,

FIG. 14 depicts a schematic diagram of an example of a provision unit.

FIG. 15 depicts a schematic diagram of an example of a training unit.

### DETAILED DESCRIPTION

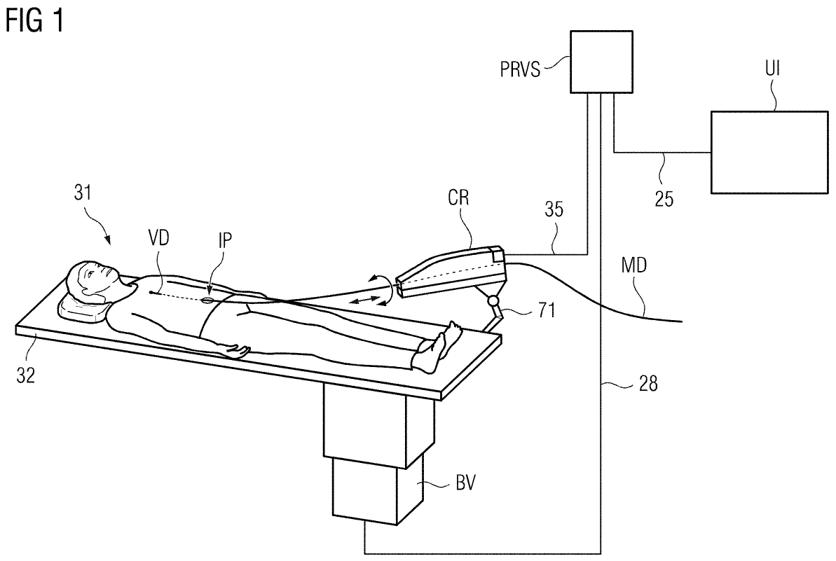

FIG. 1 shows a schematic diagram of a proposed apparatus for moving a medical object. In this figure the apparatus may have a movement apparatus CR for robotic movement of the medical object MD and a user interface UI. Moreover, the apparatus may have a provision unit PRVS.

The movement apparatus CR may be embodied as a catheter robot, in particular for remote manipulation of the medical object MD. The medical object MD may be embodied as an, in particular elongated, surgical instrument and/or diagnostic instrument. In particular, the medical object MD may be flexible and/or mechanically deformable and/or rigid at least in sections. The medical object MD may be embodied as a catheter and/or endoscope and/or guide wire. The medical object MD may further have a predefined section VD. In this case, the predefined section VD may describe a tip and/or an, in particular distal, section of the medical object MD. The predefined section VD may further have a marker structure. The predefined section VD of the medical object MD, in an operating state of the apparatus, may advantageously be arranged at least partly in an examination region of an examination object 31, in particular a hollow organ. In particular, the medical object MD, in the operating state of the apparatus, may be introduced via an introduction port at an input point IP into the examination object 31 arranged on the patient support apparatus 32, in particular into a hollow organ of the examination object 31. In this case, the hollow organ may have a vessel section in which the predefined section VD, in the operating state of the apparatus, is at least partly arranged. Moreover, the patient support apparatus 32 may be at least partly movable. For this the patient support apparatus 32 may advantageously have a movement unit BV, with the movement unit BV being able to be controlled via a signal 28 from the provision unit PRVS.

The movement apparatus CR may further be fastened by a fastening element 71, for example a stand and/or robot arm, to the patient support apparatus 32, in particular, movably. Advantageously, the movement apparatus CR may be embodied to move the medical object MD arranged therein translationally at least in a longitudinal direction of the medical object MD. The movement apparatus CR may further be embodied to rotate the medical object MD about the longitudinal direction. Additionally, or alternatively, the movement apparatus CR may be embodied to control a movement of at least a part of the medical object MD, for example a distal section and/or a tip of the medical object MD, in particular the predefined section VD. Moreover, the movement apparatus CR may be embodied to deform the predefined section VD of the medical object MD in a defined way, for example via a cable within the medical object MD.

Advantageously, the apparatus, in particular the provision unit PRVS, may be embodied to receive a dataset having an image and/or a model of the examination region. Moreover, the apparatus, in particular the provision unit PRVS, may be embodied to receive and/or to determine positioning information about a spatial positioning of the predefined section VD of the medical object MD.

The user interface UI may advantageously have a display unit and an acquisition unit. In this case the display unit may be integrated at least partly into the acquisition unit or vice versa. Advantageously the apparatus may be embodied to create a graphic display of the predefined section VD of the medical object MD based on the dataset and the positioning information. Moreover, the user interface UI, in particular the display unit, may be embodied to display the graphic display of the predefined section VD of the medical object MD with regard to the examination region based on the dataset and the positioning information.

Furthermore, the user interface UI, in particular the acquisition unit, may be embodied to acquire a user input with regard to the graphic display. In this case the user input may specify a target positioning and/or a movement parameter for the predefined section VD of the medical object MD. The provision unit PRVS may be embodied for, in particular bidirectional, communication with the user interface UI via a signal 25. In particular the user interface UI may be embodied to acquire the user input repeatedly and/or continuously. In this case, the apparatus may further be embodied to determine and/or adjust the control instruction based on the last user input acquired in each case.

The dataset may further include planning information for movement of the medical object MD. In this case the planning information may have at least one first defined area in the dataset. Moreover, the apparatus, in particular the provision unit PRVS, may be embodied, based on the positioning information and of the dataset, to identify whether the predefined section VD is arranged in the at least one first defined area, and in the affirmative case to adjust the graphic display and/or provide a recording parameter to a medical imaging device for recording a further dataset.

As an alternative or in addition the apparatus, in particular the provision unit PRVS, may be embodied to identify geometrical and/or anatomical features in the dataset. The apparatus may further be embodied, based on the identified geometrical and/or anatomical features, to define at least one second area in the dataset. Furthermore, the apparatus may be embodied, based on the positioning information and of the dataset to identify whether the predefined section VD is arranged in the at least one second defined area, and in the affirmative case to adjust the graphic display and/or provide a recording parameter to the medical imaging device for recording a further dataset. In particular the apparatus may be embodied additionally to define the at least one second defined area based on the planning information.

Furthermore, the apparatus may be embodied to determine the control instruction having an instruction for a forward movement and/or backward movement and/or rotational movement of the medical object MD based on the user input.

The apparatus, in particular the provision unit PRVS, may further be embodied to determine a control instruction based on the user input. Moreover, the provision unit PRVS may be embodied to provide the control instruction by the signal 35 to the movement apparatus CR. The movement apparatus CR may moreover be embodied to move the medical object MD in accordance with the control instruction.

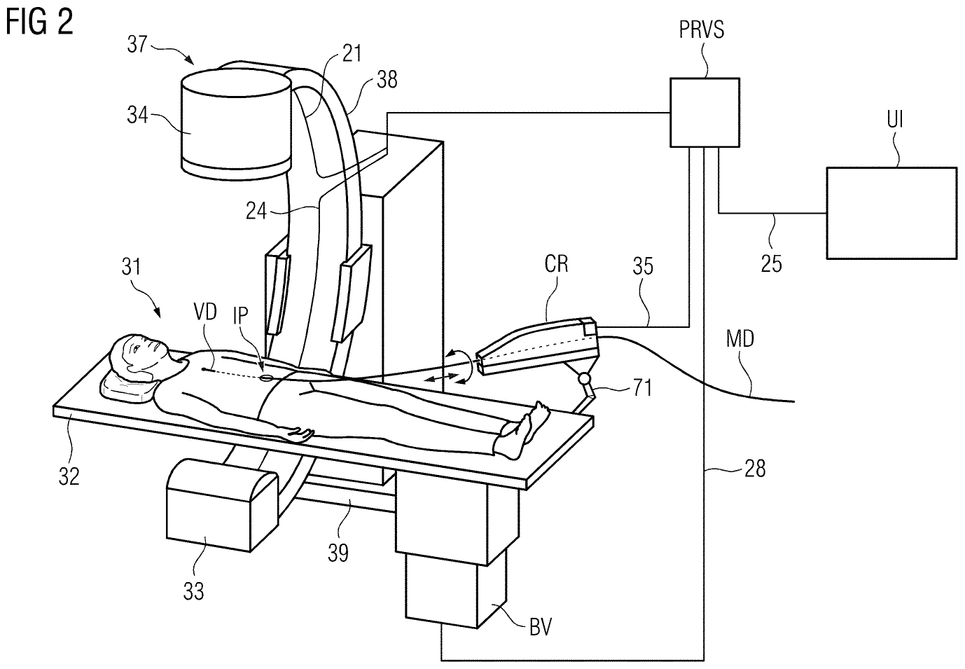

FIG. 2 shows a schematic diagram of a proposed system. In this figure, the system may have a medical imaging device, for example, a medical C-arm X-ray device 37, and a proposed apparatus for moving a medical object MD. In this case, the medical C-arm X-ray device 37 may be embodied to record the dataset having an image of the examination region of the examination object 31 and provide it to the apparatus, in particular the provision unit PRVS.

The medical imaging device in the exemplary embodiment as a medical C-arm X-ray device 37 may have a detector 34, in particular an X-ray detector, and an X-ray source 33. For recording the dataset, the arm 38 of the medical C-arm X-ray device 37 may be supported movably about one or more axes. The medical C-arm X-ray device 37 may further include a further movement unit 39, for example a wheel system and/or rail system and/or a robot arm, which makes possible a movement of the medical C-arm X-ray device 37 in space. The detector 34 and the X-ray source 34 may be fastened movably in a defined arrangement to a common C-arm 38.

The provision unit PRVS may moreover be embodied to control a positioning of the medical C-arm X-ray device 37 relative to the examination object 31 in such a way that the predefined section VD of the medical object MD is mapped in the dataset recorded by the medical C-arm X-ray device 37. The positioning of the medical C-arm X-ray device 37 relative to the examination object 31 may include a positioning of the defined arrangement of X-ray source 33 and detector 34, in particular of the C-arm 38, about one of more spatial axes.

For recording of the dataset of the examination object 31, the provision unit PRVS may send a signal 24 to the X-ray source 33. The X-ray source 33 may then emit an X-ray bundle, in particular a cone beam and/or fan beam and/or parallel beam. When the X-ray bundle, after an interaction with the examination region of the examination object 31 to be mapped, strikes a surface of the detector 34, the detector 34 may send a signal 21 to the provision unit PRVS. The provision unit PRVS may receive the dataset based on the signal 21.

Advantageously, the dataset may have an image of the predefined section VD. In this case the apparatus, in particular the provision unit PRVS, may be embodied to determine the positioning information based on the dataset.

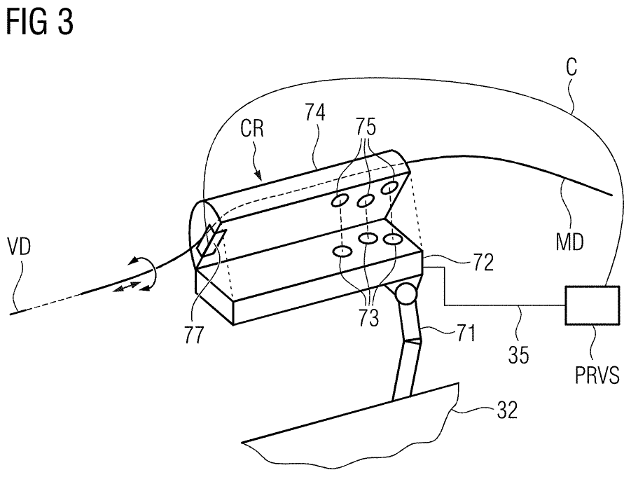

FIG. 3 shows a schematic diagram of the movement apparatus CR for robotic movement of the medical object MD. Advantageously, the movement apparatus CR may have an, in particular movable and/or drivable, fastening element 71. The movement apparatus CR may further have a cassette element 74, which is embodied for accommodating at least one part of the medical object MD. Moreover, the movement apparatus CR may have a movement element 72, which is fastened to the attachment element 71, for example a stand and/or robot arm. Moreover, the attachment element 71 may be embodied to fasten the movement element 72 to the patient support apparatus 32, in particular movably. The movement element 72 may further advantageously have at least one, for example three, actuator elements 73, for example an electric motor, wherein the provision unit PRVS is embodied for control of the at least one actuator element 73. Advantageously, the cassette element 74 may be able to be coupled, in particular mechanically and/or electromagnetically and/or pneumatically, to the movement element 72, in particular to the at least one actuator element 73. In this case, the cassette element 74 may further have at least one transmission element 75, which is movable through the coupling between the cassette element 74 and the movement element 72, in particular the at least one actuator element 73. In particular, the at least one transmission element 75 may be movement-coupled to the at least one actuator element 73. The transmission element 75 may further be embodied to transmit a movement of the actuator element 73 to the medical object MD in such a way that the medical object MD is moved in a longitudinal direction of the medical object MD and/or that the medical object MD is rotated about the longitudinal direction. The at least one transmission element 75 may have a caster and/or roller and/or plate and/or shear plate.

Advantageously, the movement element 72 may have a number of, in particular independently controllable, actuator elements 73. The cassette element 74 may have a number of transmission elements 75, in particular at least one movement-coupled transmission element 75 for each of the actuator elements 73. This enables an, in particular independent and/or simultaneous, movement of the medical object MD along different degrees of freedom of movement to be made possible.

The movement apparatus CR, in particular the at least one actuator element 73, may further be able to be controlled by the signal 35 by the provision unit PRVS. This enables the movement of the medical object MD to be controlled by the provision unit PRVS, in particular indirectly. Moreover, an alignment and/or position of the movement apparatus CR relative to the examination object 31 may be able to be adjusted by a movement of the fastening element 71. The movement apparatus CR is advantageously embodied for receiving the control instruction.

Moreover, the movement apparatus CR may advantageously have a sensor unit 77, which is embodied to detect a relative movement of the medical object MD relative to the movement apparatus CR. In this case, the sensor unit 77 may have an encoder, for example, a wheel encoder and/or a roller encoder, and/or an optical sensor, for example a barcode scanner and/or a laser scanner and/or a camera, and/or an electromagnetic sensor. For example, the sensor unit 77 may be arranged integrated at least partly into the movement element 72, in particular the at least one actuator element 73, and/or the cassette element 74, in particular, the at least one transmission element 75. The sensor unit 77 may be embodied for detecting the relative movement of the medical object MD by detecting the medical object MD relative to the movement apparatus CR. As an alternative or in addition the sensor unit 77 may be embodied to detect a movement and/or change of position of components of the movement apparatus CR, with the components being movement-coupled to the medical object MD, for example the at least one actuator element 73 and/or the at least one transmission element 74.

The apparatus, in particular the provision unit PRVS, may advantageously be embodied to determine the positioning information based on the dataset, in particular having an image and/or a model of the examination region, and based on the signal C from the sensor unit 77, in particular to determine the detected relative movement of the medical object MD with regard to the movement apparatus CR.

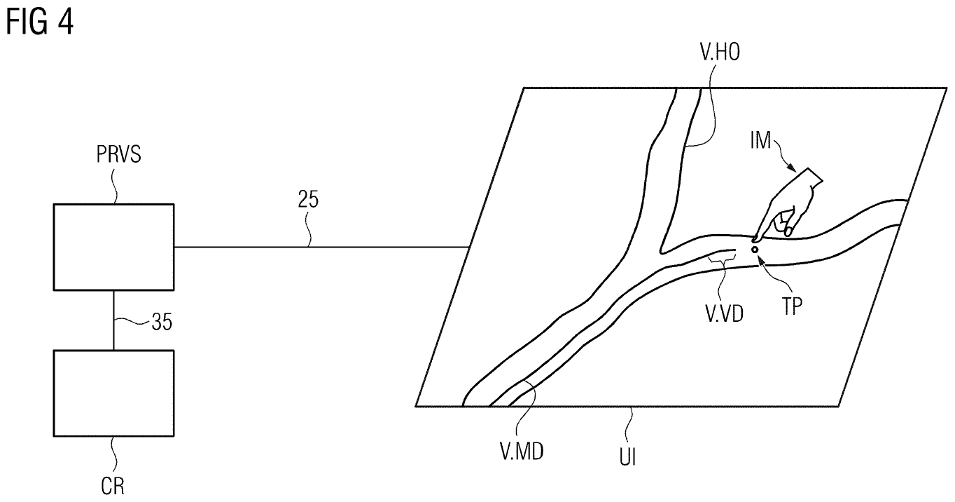

Shown schematically in FIG. 4 is the user interface UI in a form of embodiment as a touch-sensitive input display. In this figure the input display may be embodied for, in particular simultaneous display of the graphic display of the predefined section VD of the medical object MD and acquisition of the user input. The input display may advantageously be embodied as a capacitive and/or resistive input display. In this case, the input display may have a flat, touch-sensitive surface. Advantageously, the input display may be embodied to display the graphic display of the predefined section VD on the touch-sensitive surface. Moreover, the provision unit, in particular the touch-sensitive surface, may be embodied for spatially and/or temporally resolved acquisition of the user input, in particular by the input device IM, for example a finger of a user. In particular, the user interface UI may be embodied to acquire the user input including a single point input and/or an input gesture. This enables the user input advantageously to be inherently registered with the graphic display of the predefined section VD. In this case the user input may specify a target positioning TP for the predefined section VD. The graphic display may include an image and/or a model, in particular a virtual representation, of the hollow organ V.HO and/or of the medical object V.MD and/or of the predefined section V.VD.

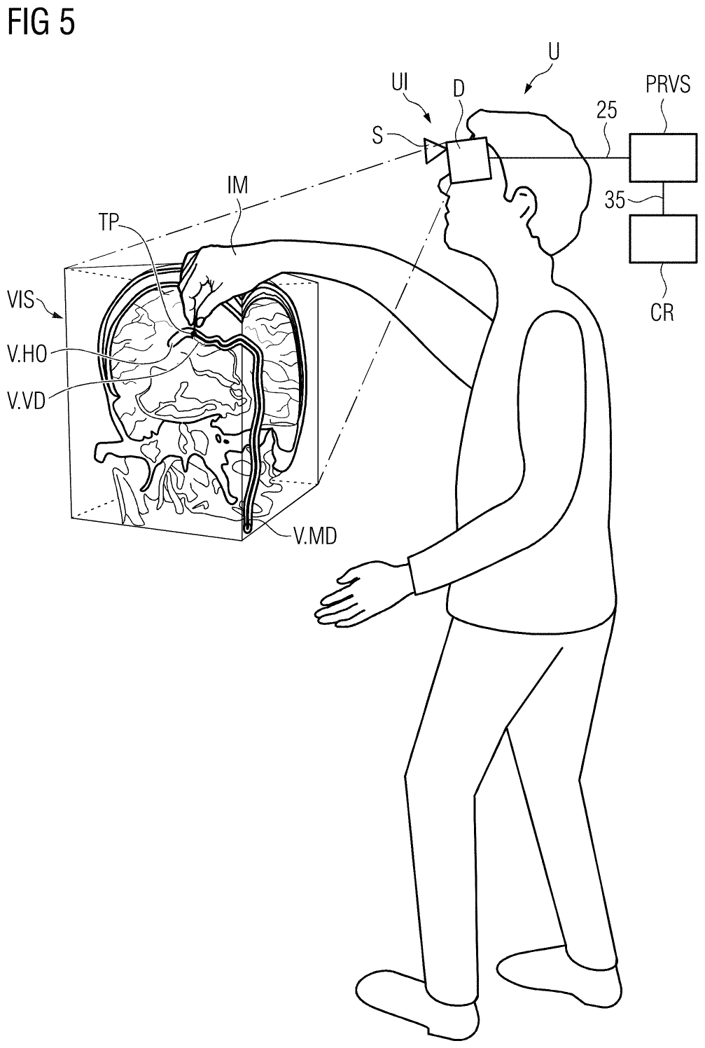

Shown schematically in FIG. 5 is a form of embodiment of the user interface UI, which is embodied to display an augmented and/or virtual reality VIS. The user interface UI in this case may have the display unit D and acquisition unit S. The display unit D may advantageously be embodied as portable, in particular able to be carried by the user U. The display unit D may further be embodied to display the augmented and/or virtual reality VIS. Advantageously the display unit D may be embodied as a data headset, which is able to be worn by the user U at least partly within their field of view.

The acquisition unit S may be embodied to acquire the user input. In this case, the acquisition unit S may be integrated at least partly into the display unit D. This enables an inherent registration between the user input and the augmented and/or virtual reality VIS to be made possible. Advantageously, the acquisition unit S may include an optical and/or haptic and/or electromagnetic and/or acoustic sensor, which is embodied to acquire the user input, in particular within the field of view of the user. In particular the acquisition unit S may be embodied for two-dimensional and/or three-dimensional acquisition of the user input, in particular based on the input device IM. The user interface UI may further be embodied to associate the user input spatially and/or temporally with the graphic display, in particular the augmented and/or virtual reality VIS. The augmented and/or virtual reality VIS may represent an image and/or include a model, in particular a virtual representation, of the hollow organ V.HO and/or of the medical object V.MD and/or of the predefined section V.VD.

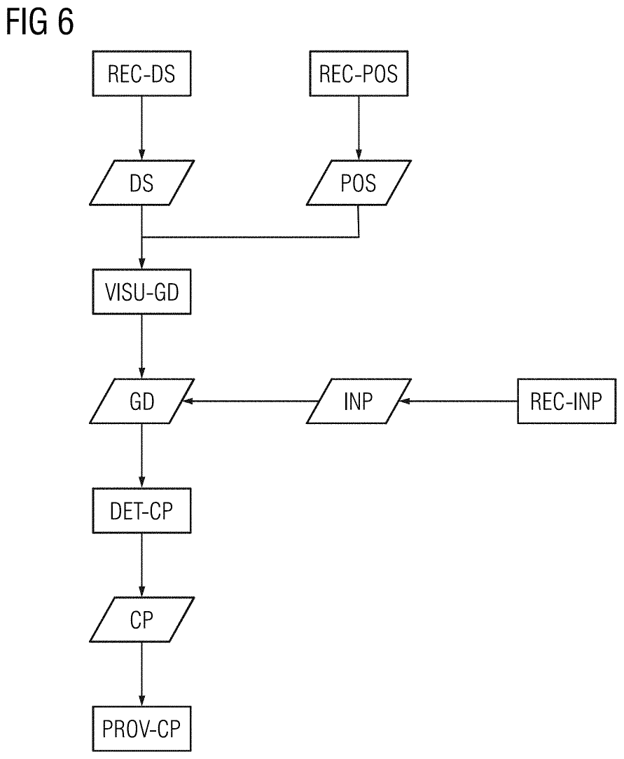

FIG. 6 shows a schematic diagram of an advantageous form of embodiment of a proposed method for providing a control instruction PROV-CP. In a first act, the dataset DS having an image and/or a model of the examination region of the examination object 31 may be received REC-DS. In this case, at least the predefined section VD of the medical object MD may be arranged in the examination area. In a second act, the positioning information POS for spatial positioning of the predefined section VD may be received REC-POS. In a third act, the graphic display GD of the predefined section VD of the medical object MD with regard to of the examination region may be displayed based on the dataset DS and the positioning information POS VISU-GD. In a fourth act, the user input INP may be acquired with regard to the graphic display GD REC-INP. In this case, the user input INP may specify a target positioning and/or a movement parameter for the predefined section VD. In a fifth act, the control instruction CP may be determined based on the user input INP, wherein the control instruction CP has an instruction for controlling the movement apparatus CR. The movement apparatus CR may be embodied to hold and/or to move the medical object MD arranged at least partly in the movement apparatus CR by transmission of a force in accordance with the control instruction CP. In a sixth act, the control instruction CP may be provided PROV-CP.

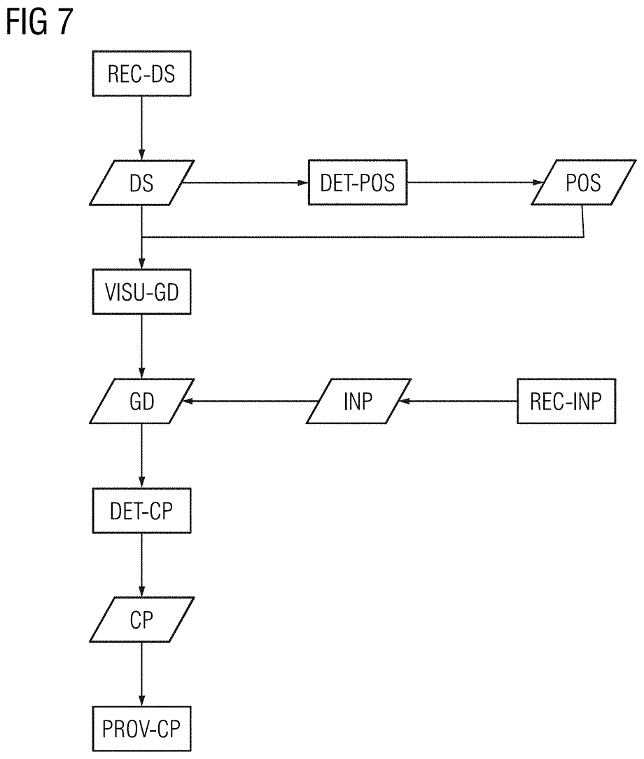

Shown schematically in FIG. 7 is a further advantageous form of embodiment of the proposed method for providing a control instruction PROV-CP. In this case the dataset DS may further have an image and/or a model of the predefined section V.VD, wherein the positioning information POS may be determined based on the dataset DS DET-POS.

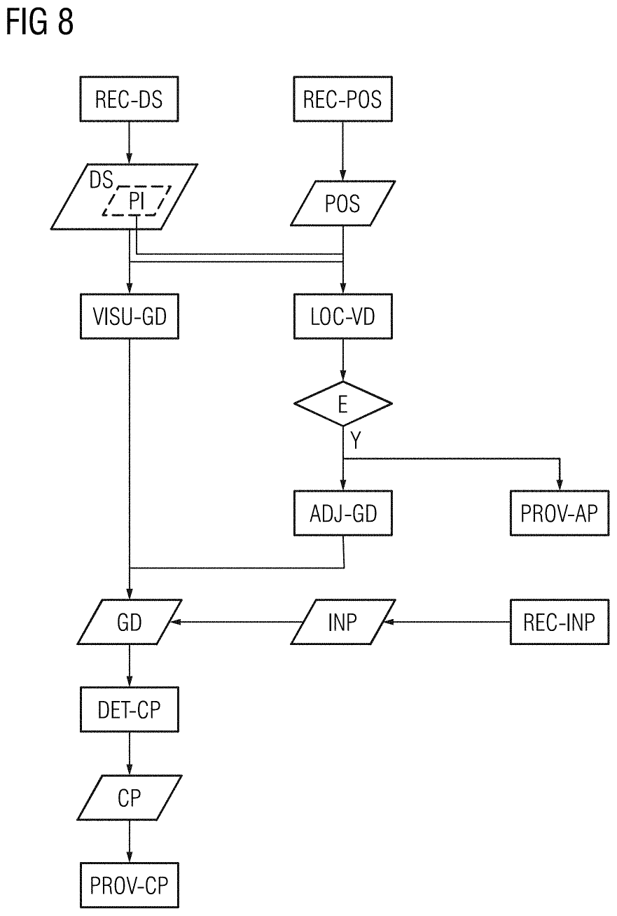

FIG. 8 shows a schematic diagram of a further advantageous form of embodiment of the proposed method for providing a control instruction PROV-CP. In this case the dataset DS may include planning information PI about a planned movement of the medical object MD, in particular of the predefined section VD. Moreover, the planning information PI may have at least one first defined area in the dataset DS. Based on the positioning information POS and of the dataset DS it may further be identified LOC-VD, whether the predefined section VD is arranged in the at least one first defined area. In the affirmative case Y, the graphic display GD may be adjusted ADJ-GD and/or a recording parameter may be provided to a medical imaging device for recording a further dataset PROV-AP.

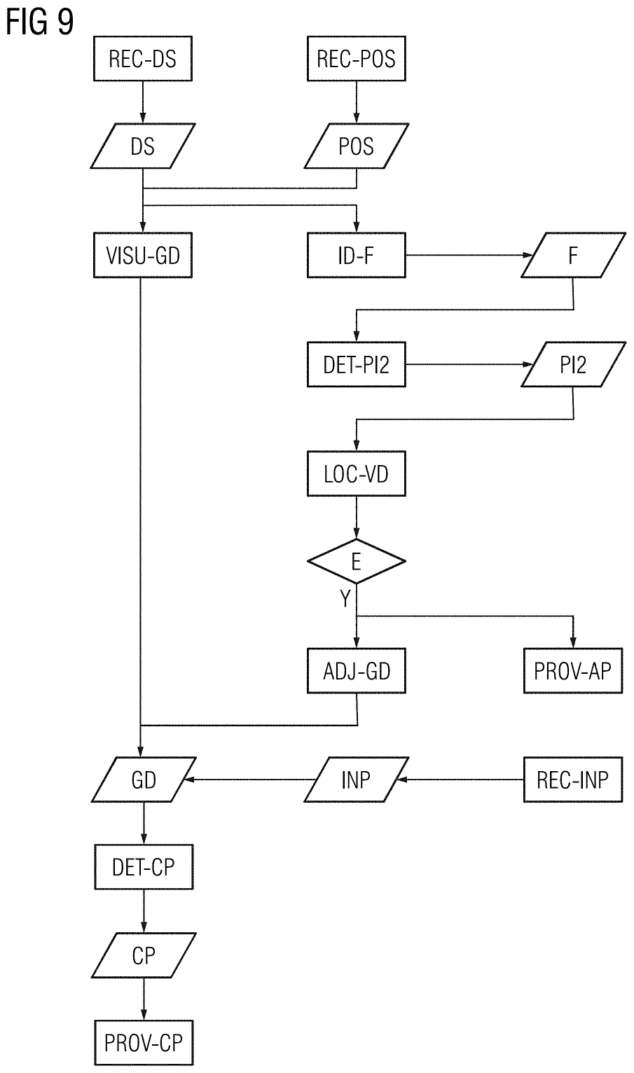

FIG. 9 shows a schematic diagram of a further advantageous form of embodiment of the proposed method for providing a control instruction PROV-CP. In this case, geometrical and/or anatomical features F in the dataset may be identified ID-F. Further based on the identified geometrical and/or anatomical features F at least one second area PI2 in the dataset DS may be determined DET-PI2. Moreover, based on the positioning information POS and of the dataset DS, it may identify LOC-VD whether the predefined section VD is arranged in the at least one second defined area PI2. In the affirmative case Y, the graphic display GD may be adjusted ADJ-GD and/or a recording parameter may be provided PROV-AP to a medical imaging device for recording a further dataset.

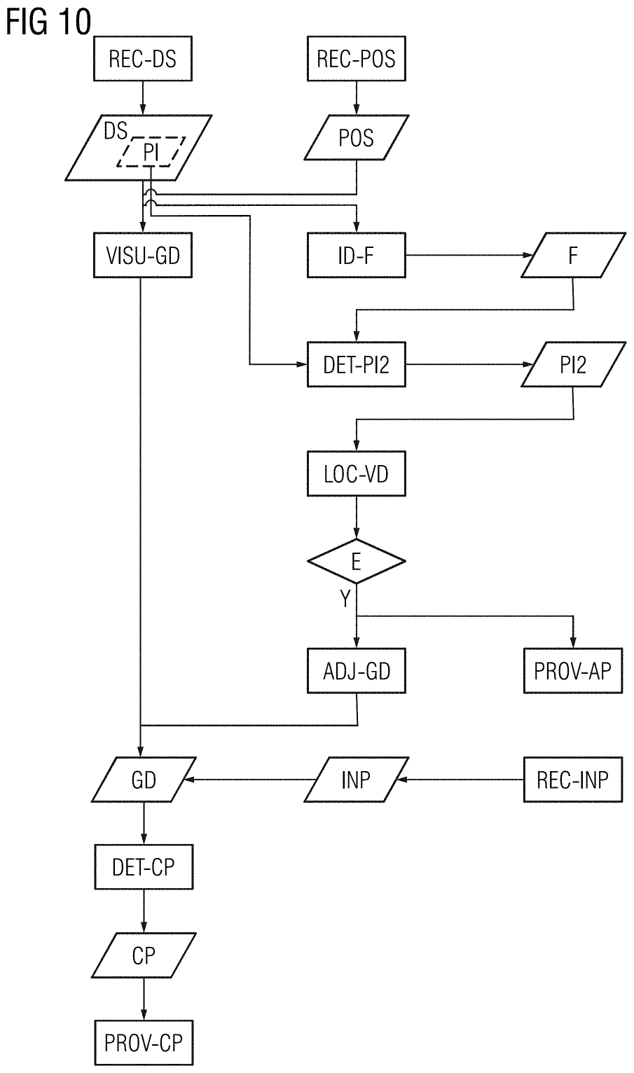

FIG. 10 shows a schematic diagram of a further advantageous form of embodiment of the proposed method for providing a control instruction PROV-CP. In this case, the dataset DS may include the planning information PI for planned movement of the medical object MD. Moreover, the at least one second area PI2 may additionally be determined DET-PI2 based on the planning information PI.

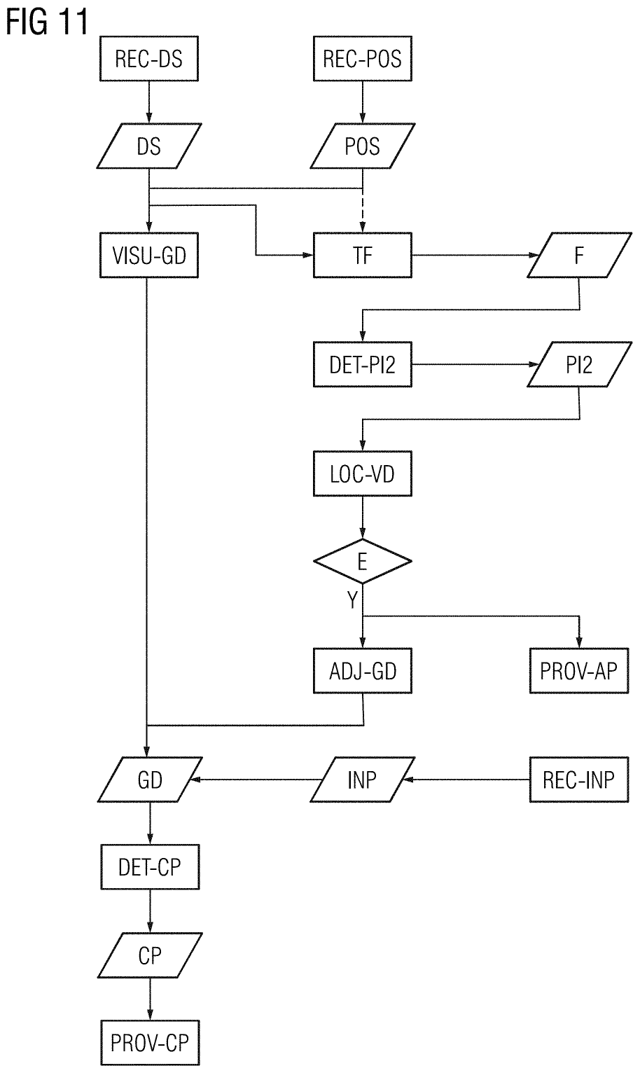

Shown schematically in FIG. 11 is a further advantageous form of embodiment of the proposed method for providing a control instruction PROV-CP. In this case the geometrical and/or anatomical features F in the dataset DS may be identified by applying a trained function TF to input data. In this case the input data may be based on the dataset DS. Moreover at least one parameter of the trained function TF may be based on a comparison of training features with comparison features. In addition, the input data may be based on the positioning information POS.

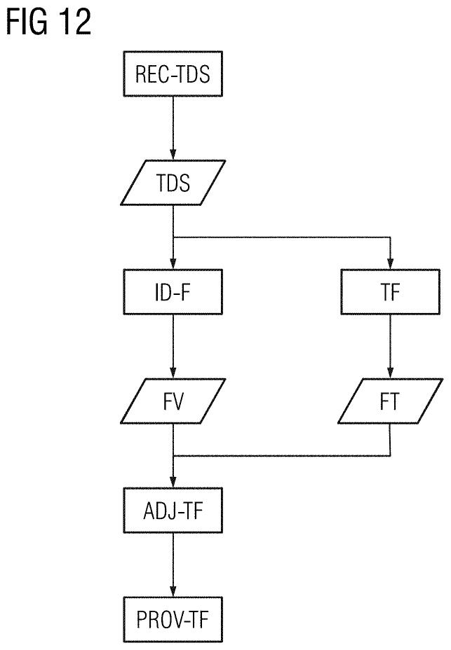

FIG. 12 shows a schematic diagram of a proposed method for providing a trained function PROV-TF. In a first act, a training dataset TDS having an image and/or a model of a training examination object may be received REC-TDS. In a second act, comparison features FC in the training dataset TDS may be identified ID-F. In a third act, training features FT may be identified by application of the trained function TF to the input data. In this case the input data may be based on the training dataset. In a fourth act, at least one parameter of the trained function TF may be adjusted ADJ-TF by a comparison of the training features FT with the comparison features FV. In a fifth act, the trained function TF may be provided PROV-TF.

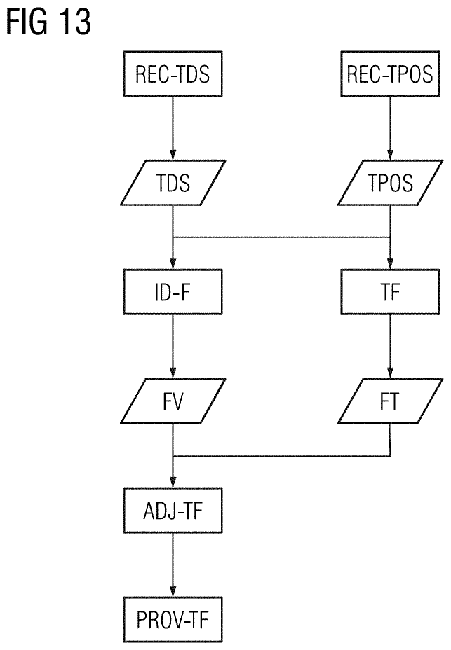

Shown schematically in FIG. 13 is a further advantageous form of embodiment of a proposed method for providing a trained function PROV-TF. In this case training positioning information TPOS for a spatial positioning of a predefined section VD of a medical object MD may be received REC-TPOS. Advantageously the predefined section VD may be arranged in the training examination area. Moreover, the input data of the trained function TF may additionally be based on the training positioning information TPOS.

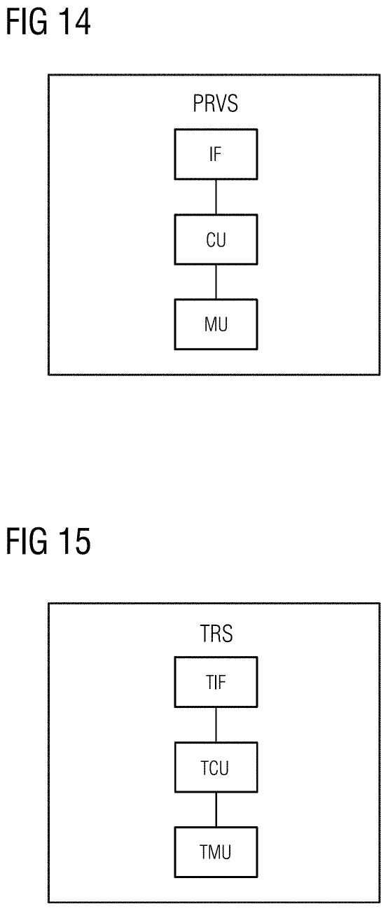

FIG. 14 shows a schematic diagram of a proposed provision unit PRVS. In this case may the provision unit PRVS may include an interface IF, a computing unit CU and a memory unit MU. The provision unit PRVS may be embodied to carry out a method for providing a control instruction PROV-CP and its aspects, by the interface IF, the computing unit CU and the memory unit CU being embodied to carry out the corresponding method acts.

FIG. 15 shows a schematic diagram of a proposed training unit TRS. The training unit TRS may advantageously include a training interface TIF, a training memory unit TMU, and a training computing unit TCU. The training unit TRS may be embodied to carry out a method for providing a trained function PROV-TF and its aspects, by the training interface TIF, the training memory unit TMU and the training computing unit TCU being embodied to carry out the corresponding method acts.

The provision unit PRVS and/or the training unit TRS may involve a computer, a microcontroller or an integrated circuit. As an alternative, the provision unit PRVS and/or the training unit TRS may involve a real or virtual network of computer (a real network is referred to as a “cluster, a virtual network is referred to as a “cloud”). The provision unit PRVS and/or the training unit TRS may also be embodied as a virtual system, which is executed in a real computer or a real or virtual network of computers (virtualization).

An interface IF and/or a training interface TIF may involve a hardware or software interface (for example, PCI bus, USB or Firewire). A computing unit CU and/or a training computing unit TCU may have hardware elements or software elements, for example, a microprocessor or a so-called FPGA (Field Programmable Gate Array). A memory unit MU and/or a training memory unit TMU may be realized as Random-Access Memory, abbreviated to RAM) or as permanent mass memory (e.g., hard disk, USB stick, SD card, Solid State Disk).

The interface IF and/or the training interface TIF may include a number of sub-interfaces, which carry out various acts of the respective methods. In other words, the interface IF and/or the training interface TIF may also be expressed as a plurality of interfaces IF or a plurality of training interfaces TIF. The computing unit CU and/or the training computing unit TCU may include a plurality of sub-computing units, which carry out various acts of the respective methods. In other words, the computing unit CU and/or the training computing unit TCU may also be expressed as a plurality of computing units CU or as a plurality of training computing units TCU.

The schematic diagrams contained in the figures described are not true-to-scale or dimensionally exact.

In conclusion, it is pointed out once again that the method described above in detail and also the apparatuses shown merely involve exemplary embodiments, which may be modified by the person skilled in the art in a wide diversity of ways without departing from the field of the disclosure. Furthermore, the use of the indefinite article “a” or “an” does not exclude the features concerned also being able to be present multiple times. Likewise, the terms “unit” and “element” do not exclude the components concerned including a number of interacting subcomponents, which where necessary may also be spatially distributed.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. An apparatus for moving a medical object, the apparatus comprising:
a movement apparatus for robotic movement of the medical object; and
a user interface,
wherein, in an operating state of the apparatus, at least one predefined section of the medical object is arranged in an examination region of an examination object,
wherein the apparatus is configured to receive a dataset having an image and/or a model of the examination region,
wherein the apparatus is configured to receive and/or determine positioning information for a spatial positioning of the predefined section of the medical object,
wherein the user interface is configured to display a graphic display of the predefined section of the medical object with regard to the examination region based on the dataset and the positioning information,
wherein the user interface is configured to acquire a user input with regard to the graphic display,
wherein the user input specifies a target positioning and/or movement parameter for the predefined section,
wherein the apparatus is further configured to determine a control instruction based on the user input, and
wherein the movement apparatus is configured to move the medical object in accordance with the control instruction.

2. The apparatus of claim 1, wherein the dataset further has an image and/or a model of the predefined section,
wherein the apparatus is further configured to determine the positioning information based on the dataset.

3. The apparatus of claim 1, wherein the apparatus is configured to determine, based on the user input, the control instruction having an instruction for a forward movement and/or backward movement and/or rotational movement of the medical object.

4. The apparatus of claim 1, wherein the user interface is configured to acquire the user input repeatedly and/or continuously, and
wherein the apparatus is further configured to determine and/or adjust the control instruction based on a last user input acquired in each case.

5. The apparatus of claim 1, wherein the user interface is configured to acquire the user input comprising a single point input and/or an input gesture.

6. The apparatus of claim 1, wherein the user interface has an input display, and
wherein the input display is configured to acquire the user input on a touch-sensitive surface of the input display.

7. The apparatus of claim 1, wherein the user interface has a display unit and an acquisition unit,
wherein the apparatus is configured to create the graphic display as augmented reality and/or virtual reality,
wherein the display unit is configured to display the augmented reality and/or the virtual reality, and
wherein the acquisition unit is configured to acquire the user input with regard to the augmented reality and/or the virtual reality.

8. The apparatus of claim 1, wherein the dataset comprises planning information for movement of the medical object,
wherein the planning information has at least one first defined area in the dataset,
wherein the apparatus is configured to identify based on the positioning information and of the dataset whether the predefined section is arranged in the at least one first defined area, and
wherein, when the predefined section is in the at least one first defined area, the apparatus is configured to adjust the graphic display and/or provide a recording parameter to a medical imaging device for recording a further dataset.

9. The apparatus of claim 1, wherein the apparatus is configured to identify geometrical and/or anatomical features in the dataset,
wherein the apparatus is configured to determine one second defined area in the dataset based on the identified geometrical and/or anatomical features,
wherein the apparatus is configured to identify based on the positioning information and the dataset whether the predefined section is arranged in the at least one second defined area, and
wherein, when the predefined section is in the at least one second defined area, the apparatus is configured to adjust the graphic display and/or provide a recording parameter to a medical imaging device for recording a further dataset.

10. The apparatus of claim 9, wherein the dataset comprises planning information for movement of the medical object, and
wherein the apparatus is configured to determine the at least one second defined area based on the planning information.

11. A system comprising:
a medical imaging device configured to record a dataset having an image of an examination region of an examination object; and
an apparatus comprising a user interface and a movement apparatus for robotic movement of a medical object,
wherein, in an operating state of the apparatus, at least one predefined section of the medical object is arranged in an examination region of an examination object,
wherein the apparatus is configured to receive the dataset from the medical imaging device,
wherein the apparatus is configured to receive and/or determine positioning information for a spatial positioning of the predefined section of the medical object,
wherein the user interface is configured to display a graphic display of the predefined section of the medical object with regard to the examination region based on the dataset and the positioning information,
wherein the user interface is configured to acquire a user input with regard to the graphic display,
wherein the user input specifies a target positioning and/or movement parameter for the predefined section,
wherein the apparatus is further configured to determine a control instruction based on the user input, and
wherein the movement apparatus is configured to move the medical object in accordance with the control instruction.

12. A method for providing a control instruction, the method comprising:
receiving a dataset having an image and/or a model of an examination region of an examination object, wherein at least one predefined section of a medical object is arranged in the examination region;
receiving and/or determining positioning information about a spatial positioning of the predefined section;
displaying a graphic display of the predefined section of the medical object with regard to the examination region based on the dataset and the positioning information;
acquiring a user input with regard to the graphic display, wherein the user input specifies a target positioning and/or a movement parameter for the predefined section;
determining a control instruction based on the user input, wherein the control instruction has an instruction for controlling a movement apparatus, and wherein the movement apparatus is configured to hold and/or to move the medical object arranged at least partly in the movement apparatus by transmitting a force in accordance with the control instruction; and
providing the control instruction.

13. The method of claim 12, wherein the dataset further comprises an image and/or a model of the predefined section, and
wherein the positioning information is determined based on the dataset.

14. The method of claim 12, wherein the dataset further comprises planning information for a planned movement of the medical object,
wherein the planning information has at least one first defined area in the dataset, and
wherein the method further comprises:
identifying, based on the positioning information and of the dataset, whether the predefined section is arranged in the at least one first defined area; and
adjusting the graphic display and/or providing a recording parameter to a medical imaging device for recording a further dataset when the predefined section is arranged in the at least one first defined area.

15. The method of claim 12, wherein geometrical and/or anatomical features are identified in the dataset,
wherein at least one second defined area in the dataset is determined based on the identified geometrical and/or anatomical features, and
wherein the method further comprises:
identifying, based on the positioning information and of the dataset, whether the predefined section is arranged in the at least one second defined area, and
adjusting the graphic display is adjusted and/or providing a recording parameter to a medical imaging device for recording a further dataset when the predefined section is arranged in the at least one second defined area.

16. The method of claim 15, wherein the dataset comprises planning information for a planned movement of the medical object, and
wherein the at least one second area is additionally determined based on the planning information.

17. The method of claim 15, wherein the geometrical and/or anatomical features in the dataset are identified by applying a trained function to input data,
wherein the input data is based on the dataset, and
wherein at least one parameter of the trained function is based on a comparison between training features and comparison features.

18. The method of claim 17, wherein the input data is additionally based on the positioning information.

19. A computer-implemented method for providing a trained function, the method comprising:
receiving a training dataset having an image and/or a model of a training examination region of a training examination object;
identifying comparison features in the training dataset;
identifying training features by application of the trained function to input data, wherein the input data is based on the training dataset;
adjustment of at least one parameter of the trained function by a comparison between the training features and the comparison features; and
providing the trained function.

20. The method of claim 19, further comprising:
receiving training positioning information for a spatial positioning of a predefined section of a medical object,
wherein the predefined section is arranged in the training examination region, and
wherein the input data is additionally based on the training positioning.
